(19) United States
(12) Patent Application Publication
(10) Pub. No.: US 2004/0254448 A1
(43) Pub. Date: Dec. 16, 2004

(54) Active therapy redefinition

(76) Inventors: Christopher Jude Amies, Walnut Creek, CA (US); Michelle Marie Svatos, Oakland, CA (US)

Correspondence Address: Elsa Keller, Legal Administrator, Siemens Corporation, Intellectual Property Department, 170 Wood Avenue South, Iselin, NJ, 08830, US

(21) Appl. No.: 10/808,215

(22) Filed: Mar. 24, 2004

Related U.S. Application Data
(60) Provisional application No. 60/457,509, filed on Mar. 24, 2003.

(51) Int. Cl. A61N 5/10

(52) U.S. Cl. 600/410

(57) ABSTRACT

A method of treating an area of interest that includes delivering a first therapeutic application to an area of interest of a patient based on an initial prescription and automatically monitoring one or more factors, exclusive of a position of the area of interest, that could affect the effectiveness of the initial prescription. Automatically modifying the initial prescription based on the automatically monitoring one or more factors and delivering a second therapeutic application to the area of interest of the patient based on the automatically modifying the initial prescription.

Applicants claim, under 35 U.S.C. § 119(e), the benefit of priority of the filing date of Mar. 24, 2003, of U.S. Provisional Patent Application Ser. No. 60/457,509 filed on the aforementioned date having the title “Active Therapy Redefinition” listing Christopher Jude Amies and Michelle Marie Svatos as inventors, the entire contents of which are incorporated herein by reference.

### BACKGROUND OF THE INVENTION

1. Field of the Invention

The present invention relates generally to a therapy devices and methods of therapy, and more particularly, to radiation therapy devices and radiation therapy methods.

2. Discussion of Related Art

Conventional radiation therapy typically involves directing a radiation beam at a tumor in a patient to deliver a predetermined dose of therapeutic radiation to the tumor according to an established treatment plan. This is typically accomplished using sources of radiation placed inside or outside the patient. An example of a radiation therapy device used to direct radiation to a patient is described in U.S. Pat. No. 5,668,847 issued Sep. 16, 1997 to Hernandez, the contents of which are incorporated herein for all purposes. When using radiation therapy devices, the amount of radiation and the placement of the radiation sources must be accurately calculated prior to commencing the radiation treatment to ensure that the physician prescribed treatment can be delivered. The expected or planned sequence of therapy delivery also varies due to changes in availability of the patient and equipment as well as possible changes in disease presentation and response to therapy. Other factors to keep in mind when using a radiation therapy device are that the patient's anatomy, physiology and clinical disposition are not static throughout the course of radiation delivery. A number of such factors are discussed below.

The radiotherapy treatment of tumors involves three-dimensional treatment volumes which typically include segments of normal, healthy tissue and organs. Healthy tissue and organs are often in the treatment path of the radiation beam. This complicates treatment, because the healthy tissue and organs must be taken into account when delivering a dose of radiation to the tumor. While there is a need to minimize damage to healthy tissue and organs, there is an equally important need to ensure that the tumor receives an adequately high dose of radiation. Thus, the goal of radiation is to administer a treatment that has a high probability of tumor control while providing an acceptably low probability of complications in normal tissue.

With new image guided and adaptive radiotherapy techniques, a wealth of information about the patient geometry is obtained, and it is desirable to use this information to tailor the treatment for complication-free tumor control at every step in the treatment. This is difficult because the three-dimensional treatment volumes for the tumor typically also include normal organs. Thus, healthy tissue and organs must be taken into account when delivering a dose of radiation to the tumor, and each type of tissue has a different type of response to varying degrees of radiation. While there is a need to minimize damage to healthy tissue and organs, there is an equally important need to choose a prescription in which the tumor receives an adequately high dose of radiation. Cure rates for many tumors are a sensitive function of the dose they receive, just as complication rates in normal organs are a function of the dose that they receive. Therefore, it is useful to have as much information as possible to understand how a certain type of tumor and certain normal structures have responded to radiation in other patients. It would be essential to monitor these quantities both during treatment during the follow up process.

Modern radiotherapy follows a process developed over the past 100 years. The process is constrained by the state of knowledge of the various diseases that include oncology and the availability of technology and experienced human resources. The general process requires that the intended radiation treatment is simulated and planned using a selection of medical images, patient measurements, physical models of radiation interaction, as well as knowledge of the disease and of the response of irradiated tissues to varying doses and dose rates. This process, largely driven by clinical trials, has produced very effective strategies of applying radiation alone or in combination with other therapies in the management of many oncology diseases. In addition, this process allows for changes in current clinical practice only with conclusive evidence of patient benefit, preferably supported by clinical trails. Trials are constrained by access to expert staff, advanced technology and appropriate patients. Thus, the process often is unable to be readily modified in response to new information. The process has by necessity led to the concepts of image guided therapy and dose limited and optimized radiation delivery.

In recent years there have been significant technological and scientific developments in the fields of physiological, biological and treatment imaging. These developments enable clinicians and scientists to better define oncology diseases and more accurately deliver therapies. Radiation exposure (and thus treatment) is fundamentally defined by molecular changes. Thus, biological targets, measures of dose and biological response to radiation are eventually determined and monitored using images of molecular activity. The new and rapidly expanding field of molecular imaging will have a significant impact on the future management of oncology diseases. As such, it must be considered in developing a vision for future oncology processes.

Another factor that adds complexity to the planning process is the fact that many organs change size, shape and position from day to day. This also affects the prescription because margins must be added to these structures to account for the likely extent of the changes.

A better understanding of the likely effect of these factors could result in a more accurate plan and higher probability of complication free tumor control.

Several processes have been proposed in the past to take into account a number of the factors discussed previously. In particular, processes known under the guise of Adaptive Radiation Therapy attempt to change a treatment plan based on measurements of dose delivery to a target area and/or images of the target area. Adaptive Radiation Therapy is a closed loop radiation process by systematically monitoring the target area and using such monitoring to re-optimize the treatment plan.

A simplified Adaptive Radiation Therapy process is shown in FIG. 1. As schematically shown, the Adaptive Radiation Therapy process 10 includes taking an image of the target area per step 12 and defining a prescription 14, a radiation treatment plan 16 and positioning the target area in a radiation field per step 18. Once the treatment plan 16 is established and the target area is positioned per step 18, radiation is delivered per step 20 in accordance with the treatment plan 16. After delivery of the radiation is complete, a verification process is performed per step 22 where an image of the target area during delivery of the radiation is taken and compared with the image taken in step 12. Based on the comparison, the target area and patient are repositioned via an image guided patient positioning process 24 and the treatment plan is altered via an image guided radiation therapy process 26. This process is repeated until the treatment is complete.

One disadvantage of the above described Adaptive Radiation Therapy process is that it relies on a single set of image data to control the treatment plan and does not take into account other factors, such as daily anatomical changes in position and size of target area, changes in physiological functions of the target area, changes in availability of the patient or radiation used in the treatment.

Adaptive radiotherapy (ART) was first proposed to account and correct for the motion of the target tissues, during the course of radiation treatment. It is generally considered a way of imaging while treating, and potentially correcting for motion related to anatomical changes during radiation delivery. In this form, adaptive radiotherapy extends the concepts of image guidance and dose optimization to a natural technical limit. In its full form adaptive radiotherapy presents major challenges in technology development and significant hurdles for general clinical acceptance.

Others have proposed methods of adaptive radiotherapy treatment on the basis of changes in anatomical images. General adaptive radiation therapy approaches stress the importance of planning the therapy just prior to delivery.

### SUMMARY OF THE INVENTION

One aspect of the present invention regards a method of treating an area of interest that includes delivering a first therapeutic application to an area of interest of a patient based on an initial prescription and automatically monitoring one or more factors, exclusive of a position of the area of interest, that could affect the effectiveness of the initial prescription. Automatically modifying the initial prescription based on the automatically monitoring one or more factors and automatically delivering a second therapeutic application to the area of interest of the patient based on the automatically modifying the initial prescription.

A second aspect of the present invention regards a method of active therapy redefinition that includes performing a diagnosis process on a patient and automatically delivering a first dose of therapeutic radiation to an area of interest of the patient based on the diagnosis process. Automatically monitoring one or more factors, exclusive of a position of the area of interest, that could affect the effectiveness of the automatically delivering the first dose of therapeutic radiation to the area of interest of the patient based on the diagnosis process. Automatically calculating a second dose of therapeutic radiation based on the automatically monitoring one or more factors; and automatically delivering the second dose of therapeutic radiation to the area of interest based on the automatically calculating.

Each aspect of the present invention may provide the advantage of taking into account one or more factors that can be used to improve a patient's treatment plan.

Each aspect of the present invention may provide the advantage of providing a therapy framework that allows useful therapy components to be developed and to mature in isolation.

Each aspect of the present invention may provide the advantage of enabling the testing of several underlining hypothesis in parallel and the rapid evolution of clinical practice without sacrificing the principle of evidence based medicine.

Further characteristics and advantages of the present invention ensue from the following description of exemplary embodiments by the drawings.

### BRIEF DESCRIPTION OF THE DRAWINGS

FIG. 1 schematically illustrates an Adaptive Radiation Therapy process;

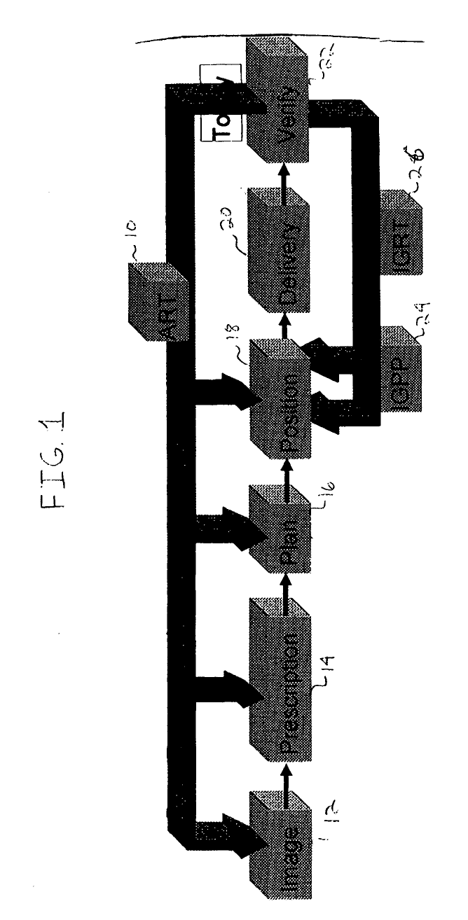

FIG. 2 shows an embodiment of a radiation therapy machine in accordance with the present invention; and

FIG. 3 shows a flow chart of a mode of treating an area of interest with radiation in accordance with the present invention.

### DETAILED DESCRIPTION OF THE PREFERRED EMBODIMENTS

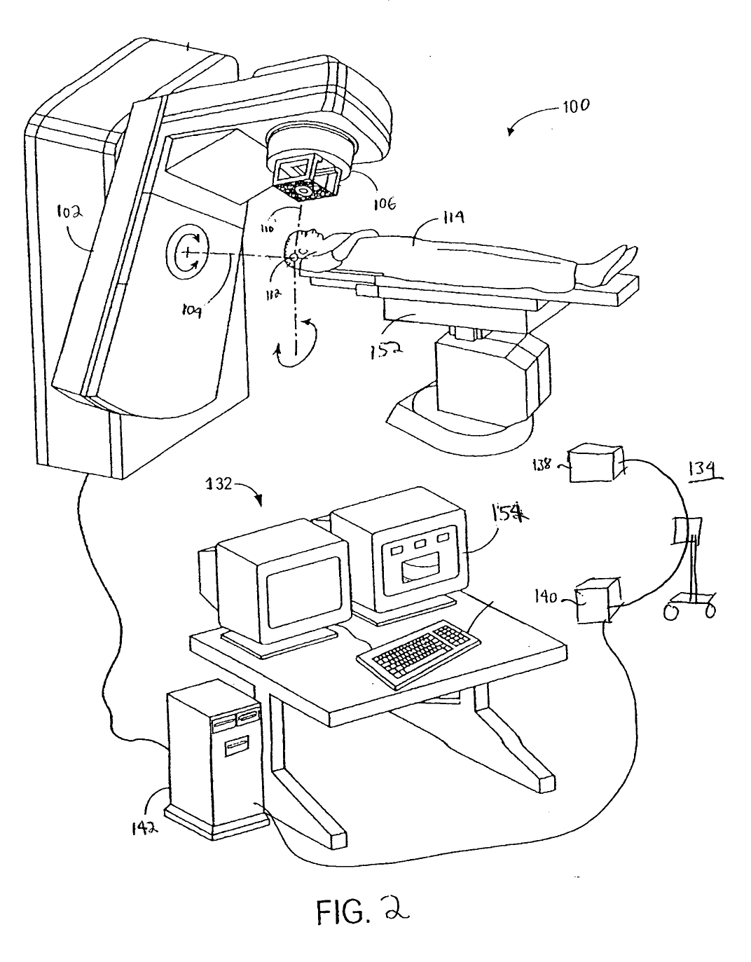

A radiation therapy machine 100 that employs a therapy approach in accordance with the present invention is shown in FIG. 2. The radiation therapy machine 100 includes a gantry 102 which can be swiveled around a horizontal axis of rotation 104 during the course of a therapeutic treatment. A beam source 106 is used to generate radiation beams in any of a number of ways well-known to those skilled in the art. For example, the beam source 106 may include a dose control unit 108 used to control a trigger system. The trigger system generates injector trigger signals that are fed to an electron gun in a linear accelerator (not shown) located inside the gantry 102 to produce the high energy radiation, such as an electron beam or photon beam, required for the therapy. The beam source 106 may include separate sources of radiation for photons and electrons. The axis of the radiation bundle emitted from the linear accelerator and the gantry 102 is designated by beam path 110.

During a course of treatment, the radiation beam is trained on treatment zone 112 of an object 114, for example, a patient who is to be treated and whose tumor lies at the isocenter of the gantry rotation. Several beam shaping devices are used to shape radiation beams directed toward the treatment zone 112. In particular, a multileaf photon collimator and a multileaf electron collimator may be provided as disclosed in U.S. patent application Ser. No. ______, filed on Mar. 12, 2003 Siemens Case No. 2002P13825US), the entire contents of which are incorporated herein by reference.

Note that while the radiation therapy machine 100 described above has the capability of providing either photon beam or electron beam treatments, the present invention is equally applicable to radiation therapy machines that have only one radiation source.

Radiation therapy machine 100 also includes a central treatment processing or control unit 132 that controls an active therapy redefinition process in accordance with the present invention that will be discussed in detail below. The active therapy redefinition process applied to a patient is based in part on images taken of the patient as part of either a diagnosis process or a simulation process. In either process, one or more three-dimensional (3D) image volumes of the patient are taken in a well known manner, such as magnetic resonance imaging, CT or x-ray imaging. The 3D image volume can be taken at a different site, such as a CT simulation site or a diagnostic center, or may be taken at the site of the radiation therapy machine 100 by an imaging system, such as a portal imaging system or photon imager 134 or a magnetic resonance imaging system 136 that is attached to an end of the gantry 102 as shown in FIG. 2. In the case of an x-ray imaging system, an imaging radiation source 138, such as an x-ray source, is attached to an end of the gantry 102 so as to face the imaging system 134 and subjects the treatment zone 112 to radiation that is imaged by detector 140 of the imaging system 134 in a well known manner. The image information is then fed from the imaging system 134 to a computer 142 of the central treatment processing unit 132.

As mentioned above, the radiation therapy machine 100 and an imaging system 134, 136 are used, in conjunction with a variety of software tools to be described below, to perform active therapy redefinition in accordance with the present invention, which is a comprehensive approach to therapy that includes the possibility to modify the prescription, not just the delivery of prescribed therapy. Thus, the previously discussed process of applied radiation therapy is a subset of active therapy redefinition.

Before going into detail as to the active therapy redefinition process of the present invention, a brief review of the basics of the process will be undertaken. In particular, active therapy redefinition focuses on the fact that the diagnosis (description of the disease state and patient condition) and the clinical prescription (a description of clinical intent, goals and constraints to the choice of therapy) are the key drivers of all processes associated with patient therapy management. Furthermore, the disease state is dynamic, requiring monitoring, such as via lab tests and imaging, and the possibility of redefinition throughout a course of therapy. The prescription must also be reviewed via lab testing and imaging and reevaluated due to limitations in delivery, the patient's tolerance to the therapy and the clinical response to therapy. Finally, images and dose are just two of many enablers associated with therapy processes and should be used appropriately and in conjunction with all relevant data.

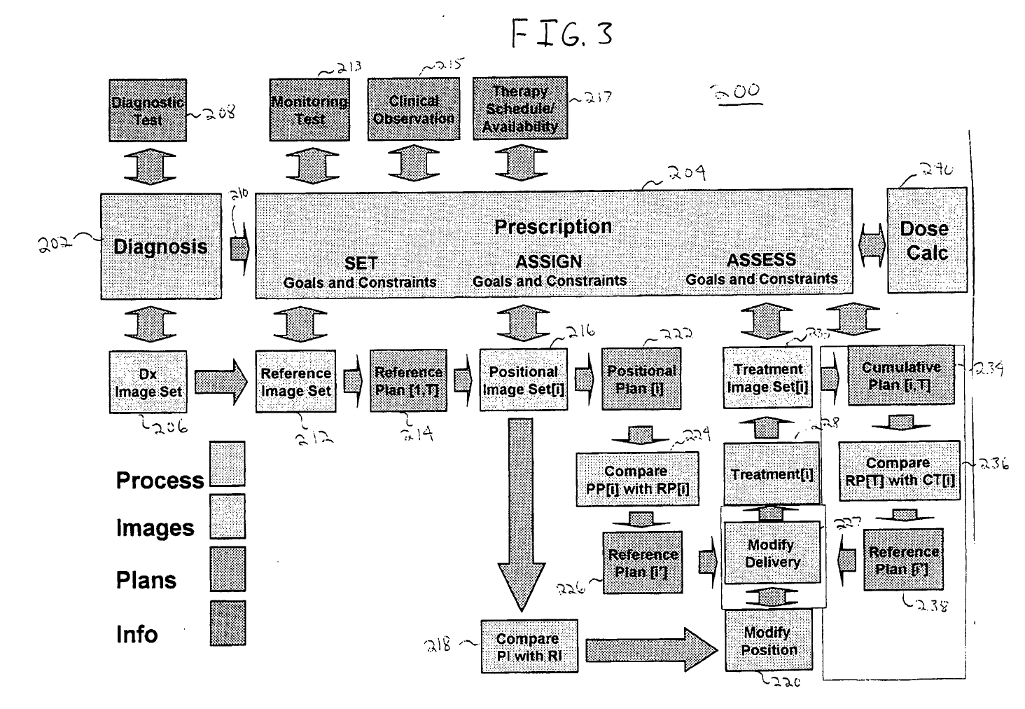

Active therapy redefinition requires a complex model of the patient and therapy to prescribe and deliver a radiotherapy treatment. An example of an active therapy redefinition technique is shown in FIG. 3. As shown, the active therapy redefinition technique 200 includes a diagnosis process 202 and a therapy prescription process 204. The diagnosis process 202 performs an analysis of all relevant information used to ascertain the disease state and general patient condition. Inputs to the diagnosis process 202 include diagnostic image set(s) 206 relevant to the patient's disease generated by imaging systems 134, 136 and the results of diagnostic tests 208 that are well known in the art. Note that in the description to follow the various tests, measurements and observations performed in steps 206, 208, 213, 215 and 217 that are inputs for the processes 202, 204 can be performed manually or preferably automatically when feasible to do so. Furthermore, the various processes and plans 202, 204, 212, 214, 216, 218, 220, 222, 224, 226, 227, 228, 234, 236, 238 are performed in an automatic manner upon receipt of the various inputs 206, 208, 213, 215 and 217 as mentioned above and described below.

During the diagnosis process, decisions are made concerning the type and extent of disease and thus ‘value’ is added, via clinical judgment, to the diagnostic image set(s) 206 which may be transfered to the therapy prescription activity in order to influence the definition of disease state, stage of the disease and extent of the disease prior to, and potentially during, therapy. The use of such diagnostic data in prescribing a treatment therapy is represented by arrow 210.

As shown in FIG. 3, the therapy prescription process 204 performs three major functions: 1) sets goals and constraints for the therapy prescription; 2) assign goals and constraints for the therapy prescription; and 3) assess goals and constraints for the therapy prescription. The therapy process begins by taking from the diagnostic value added image set(s) 206 a reference image set 212 that includes images of the target volume and associated sensitive structures of the patient anatomy in the treatment position. The image set 212 represents a static image of the anatomy as it presented on one occassion prior to any treatment. This image set 212 will become the ‘gold standard’ patient information upon which the first round of treatment planning will be based. Note that examples of goals and constraints for the therapy prescription are: total dose, dose per fraction, a fractionation schedule, identification of whether treatment is complete, definition of anatomical stuctures associated with a disease as well as organs that are not to be unduly irradiated and definition of an anatomical point to be irradiated to a required minimum dose.

The image set 212 generally will be a set of axial computed tomography slices, which is then reconstructed into a 3D volume. The reconstructed 3D volume is a geometrically accurate model of the patient anantomy that is made up of x-ray attenuation coeficients for all tissues in the volume. Future implementation of adaptive therapy redefinition may be based on other image modalities, such as MR or PET, or these modalities may be fused with the reference image set (either manually or automatically at acquisition time in the case of the biograph, for example) for more precise targeting information.

During the therapy prescription process 204, the clinician interacts with the ‘reference image set’ by setting clinical goals and contraints that define a course of radiotherapy. This is a complex and frequently iterative process, that will be specific to the patient's disease type and medical condition. At a high level, it will define the target volume, total dose, dose per fraction, the fractionation schedule and whether this is a complete treatment or a component (boost) of a broader course of radiation treatment. It may include the definition of anatomical stuctures associated with the disease as well as organs that must not be unduly irradiated if the patient is to tolerate a tumoricidal dose. It will include the definition of an anatomical point(s) to be irradiated to a required minimum dose. Internationally defined concepts of gross tumor volume (GTV), clinical target volume (CTV) and planning target volume (PTV) are associated with defining tissue volumes to be irradiated. General dose limits are often defined in the prescription. These may be point doses or repesented by a dose volume histograms. Note that possible inputs for modifying the therapy prescription process 204 include monitoring test results 213, clinical observations 215 and patient attendance or machine breakdowns 217.

The therapy prescription process 204 also defines technical aspects of the proposed therapy delivery. This includes the choice of radiation type and energy, the selection of beams and relative weighting that may imply intensity modulation. As shown in FIG. 3, the reference image set 212 is used to generate a reference plan 214 that includes reference image, anatomical points and volumes, and dose or dose distribution. Note that the reference plan 214 can be generated by a variety of well known methods that may use known treatment planning software with a dose calculation enging and possibly an inverse dose optimization method. The reference plan 214 indicates what is dosimetrically expected for the first fraction of radiation and the total course of radiation delivery. The reference plan 214 has relevance for the first fraction [1] of therapy delivered on the first day of treatment and for the total course [T] of the treatment delivered on the Tth day of treatment which is represented by the nomenclature Reference Plan [1, T] in FIG. 3.

In a manner similar to that for the reference image set 212, a positional image set 216 of the patient is generated at the radiation source 106, such as a linear accelerator. The positional image set 216 may be generated in a number of known ways, such as using the images from the portal imaging system and performing The positional image set 216 is either two or more two dimensional images or, preferably, a volume set of three dimensional data, similar to the reference image set 212 that includes information of the actual treatment isocenter and a model of the machine mechanical characteristics taken on the ith day of treatment (i=1, . . . T). Gathering such information related to the position of the treatment isocenter can be difficult in view of the realities of patient motion and constraints of set-up. Despite the difficulty in gathering the information, it nonetheless can be a useful subset of data relevant to strategies aimed at achieving the goals for a particular disease site and clinical intent guided by the prescription process.

The derived positional image set 216 is used by the therapy prescription process 204 to assign goal and constraints for the therapy. For example, the positional image set 216 can be processed in known ways to outline or contour the target and/or the sensitive structures or identify the isocenter of treatment. In addition, the positional image set 216 is compared with the reference image set 212 per step 218. Such comparison may as a minimum confirm that it is reasonable to proceed or more precisely assess the impact of this particular position to the total course of the therapy program, or flag a potential danger requiring more immediate attention. As shown in step 220, the comparison can lead to identifying an offset that is used to modify the patient position. This offset is used to move the patient or the table is moved or the machine settings are adjusted to overcome the offset and align the target area and the radiation source.

The positional image set 216 can also be used to replan the therapy or re-optimizing and creating a new or modified ‘reference plan’ that better represents the prescribed course of therapy. In particular, the positional image set 216 contains positional information regarding the target area and surrounding sensitive areas from the 1 through ith days of treatment and so the reference plan can be modified to take into account the movement of the target area and surrounding areas as a function of time. If performed in real time, for each fraction this would be the optimal ‘reference plan’ for the fraction. If performed ‘off line,’ the new or modified ‘reference plan’ would take into account what was delivered in the past to determine the optimal ‘reference plan’ to best realize the intended course. It is possible that this could include predictions of patient motion in subsequent fractions.

As shown in FIG. 3, the new or modified reference plan is determined by first determining a positional plan 222 from the positional image set 216 in a number of known ways, such as using a known radiation therapy plan or by the clinician using his or her own knowledge to determine the positional plan. The positional plan 222 is the result of the interaction of the therapy prescription process 204 with the positional image set 216 by assigning the proposed goals and constraints. The generated positional plan 222 defines the dose volume statistics that would be delivered given the current relationship between imaged anatomy and proposed radiation beams. This data is specific to this fraction of therapy, but relevant to the complete course.

Ideally the positional plan 222 is created prior to treatment delivery, but it could also be recorded as a better representation of what has been delivered if compared to the reference plan 214 per step 224. The process of comparing plans generates an error reference that can be used to modify the delivery plan (beams and weighting of the importance of each beam) per step 227 and/or the patient position (via an offset in the alignment of the target area) per step 220. This may as a minimum confirm that it is reasonable to proceed or more precisely assess the impact of this fraction to the total course as fractions accumulate, or flag a potential danger requiring more immediate attention.

The comparison process per step 224 also includes the possibility of re-planning the therapy or re-optimizing and creating a new or modified reference plan per step 226 that better represents the prescribed course of therapy. If performed in real time, for each fraction this would be the optimal reference plan for the fraction. If performed ‘off line’ the new or modified reference plan takes into account what was delivered in the past to determine the optimal reference plan to best realize the intended course. It is possible that this could include predictions of patient motion in subsequent fractions.

After it is decided whether or not to modify the patient position and/or the treatment delivery per steps 220 and 227, a treatment process 228 is defined and performed. This process is associated with the delivery technique such as the implementation of intensity modulation and the level of delivery automation.

During the delivery of radiation during the treatment process 228, an image of the patient being treated can be generated per step 230 via imaging system 134 and/or 136. This could be a complete or partial volume, image set 230. A record of simple patient motion during therapy and possibly an image of the exit dose for each radiation field may be included.

The prescription process can than be activated to assess the goals and constraints of the therapy prescription 204. This can be performed in ‘real time’ as treatment is delivered or ‘off line’ after treatment is completed. This interaction with the treatment image and prior data sources enables the generation of a cumulative treatment plan 234 that is a document of what has been delivered and can thus form part of a tool used to determine what is required in order to complete the prescribed course of therapy. A process 236 is thus defined that compares the cumulative treatment plan 234 with the reference plan 214 for the course. This process generates a modified reference plan 238 that guides subsequent fractional treatment delivery. The modified reference plan 238 can be generated in a number of known ways.

As shown in FIG. 3, a dose calculation process 240 interacts with the therapy prescription process 204 and cumulative plan 234 to assign a theoretical dose to the reference image set 212. The theoretical dose is determined in a well known manner, such as by a radiation therapy plan/process or based on personal knowledge of the clinician

An example of an active therapy redefinition process for treating an area of interest will be described below. In particular, a patient diagnosed as a candidate for radiation therapy is subjected to a variety of tests, such as the diagnostic tests 208, monitoring tests 213, clinical observations 215 and diagnostic images 206, prior to delivering the first (i=1) therapeutic application of a first dosage of radiation to an area of interest to be treated in the patient, such as a tumor. After the diagnosis process 202 and the prescription process 204 receive the results of the initial battery of tests given to and the images taken of the patient, a diagnosis is established per process 202 which is fed to process 204 so that an initial therapeutic prescription (i=1) is determined for the patient. Such initial prescription is determined by taking into account by defining clinical intent, goals and constraints of the treatment for the area of interest.

At this stage, the patient is placed on a treatment table 152 and is positioned per step 220. Based on the initial therapeutic prescription and the diagnosis process 202, a first therapeutic application of a first dose of therapeutic radiation is delivered to the area of interest. After the first dose is delivered, then the patient leaves the treatment area and returns at a later time to undergo a second treatment (i=2) pursuant to the initial therapeutic prescription. However, prior to the application of the second treatment, the patient undergoes automatic or manual monitoring via processes 206, 208, 213, 215, 217 of one or more factors, exclusive of a position of the area of interest, that could affect the effectiveness of the initial prescription. Such monitoring can be performed during the application of the first dose or after the first dose but prior to a second dose. The monitoring includes laboratory testing, physiological measurement, clinical observation of the patient and imaging the area of interest via CT imaging or magnetic resonance imaging. In addition, such one or more factors include anatomical and physiological variations, stage of disease, stage of treatment, changes in applying the first and second therapeutic applications due to unscheduled breaks in the treatment, within the area of interest.

After the monitoring is performed on the patient, the initial prescription is modified based on the monitoring of the one or more factors. The modified prescription includes automatically calculating a second dosage based on the monitoring of the one or more factors. Next, the patient is placed on the treatment table 152 and is positioned pursuant to the modified initial prescription. After being properly positioned, a second therapeutic application of a dose of radiation is delivered to the area of interest based on the modified initial prescription and the calculated second dosage. The above process is then repeated until the treatment process is completed.

Note that the process described above with respect to FIG. 3 is not constrained to any particular time scale. There may be days that elapse between the execution of two adjacent boxes shown in FIG. 3 in some instances and only seconds between others, depending upon the implementation chosen. Furthermore, although the arrows show the initial order of events, the entire process may be re-initiated if new input (images or information) is admitted to the process, since the effects of new input could require modifications throughout the process in order to ensure that the prescribed therapy is delivered as intended. In addition, the described processes of comparing images or plans do not need to be performed in real time and can be performed as it is convenient and relevant for a particular disease type. The processes of comparing of images or plans also can be performed off line in a work flow optimized manner as variations in the delivered therapy are accommodated across subsequent treatment episodes, by modification of the prescription.

In summary, the present invention regards an Active Therapy (Delivery) Redefinition (ATR) approach to therapy that incorporates into the delivery process changes in anatomical and physiological patient data or other patient related information that could influence the original clinical intent of the treatment. This includes clinical observation laboratory results or imaging experiments. This approach ensures that the physician's prescription is delivered but includes the possibility of prescription modification throughout the course and subsequent courses of therapy.

Those skilled in the art will appreciate that various adaptations and modifications of the just described preferred embodiments can be configured without departing from the scope and spirit of the invention. Therefore, it is to be understood that, within the scope of the appended claims, the invention may be practiced other than as specifically described herein.

We claim:

1. A method of treating an area of interest, the method comprising:
delivering a first therapeutic application to an area of interest of a patient based on an initial prescription;
automatically monitoring one or more factors, exclusive of a position of said area of interest, that could affect the effectiveness of said initial prescription;
automatically modifying said initial prescription based on said monitoring one or more factors; and
delivering a second therapeutic application to said area of interest of said patient based on said automatically modifying said initial prescription.

2. The method of claim 1, wherein said area of interest includes a tumor.

3. The method of claim 1, wherein said first therapeutic application comprises a dose of radiation delivered to said area of interest.

4. The method of claim 1, wherein said one or more factors comprise anatomical and physiological variations within said area of interest.

5. The method of claim 1, wherein said one or more factors comprise a stage of disease within said area of interest.

6. The method of claim 1, wherein said one or more factors comprises a stage of treatment of said area of interest.

7. The method of claim 1, wherein said monitoring comprises imaging said area of interest.

8. The method of claim 1, wherein said monitoring comprises laboratory testing of said patient.

9. The method of claim 1, wherein said monitoring comprises physiological measurement of said patient.

10. The method of claim 1, wherein said monitoring comprises clinical observation of said patient.

11. The method of claim 1, wherein said one or more factors comprises changes in applying said first and second therapeutic applications due to unscheduled breaks in said method of treatment.

12. The method of claim 1, wherein said automatically monitoring is performed during said delivering of said first therapeutic application.

13. The method of claim 1, wherein said automatically monitoring is performed after said delivering said first therapeutic application and prior to said delivering said second therapeutic application.

14. The method of claim 7, wherein said imaging comprises taking a magnetic resonance image of said area of interest.

15. The method of claim 7, wherein said imaging comprises taking a CT image of said area of interest.

16. The method of claim 1, further comprising defining said first therapeutic by defining clinical intent, goals and constraints of said method of treating said area of interest.

17. A method of active therapy redefinition, comprising:
performing a diagnosis process on a patient;
automatically delivering a first dose of therapeutic radiation to an area of interest of said patient based on said diagnosis process;
automatically monitoring one or more factors, exclusive of a position of said area of interest, that could affect the effectiveness of said automatically delivering said first dose of therapeutic radiation to said area of interest of said patient based on said diagnosis process;
automatically calculating a second dose of therapeutic radiation based on said automatically monitoring one or more factors; and
automatically delivering said second dose of therapeutic radiation to said area of interest based on said automatically calculating.

18. The method of claim 17, wherein said diagnosis process comprises analyzing relevant information regarding a disease state and a condition of said patient.

19. The method of claim 17, further comprising generating at least one image set relevant to said area of interest of said patient and applying said at least one image set to said performing said diagnosis process.

20. The method of claim 17, wherein said performing said diagnosis process comprises performing decisions concerning the type and extent of disease within said area of interest.

21. The method of claim 19, further comprising:
automatically performing a therapy prescription process that comprises said automatically calculating said second dose of therapeutic radiation;
wherein said performing said diagnosis process comprises performing decisions concerning the type and extent of disease within said area of interest; and
wherein value is added to said at least one image set and said value added to said at least one image set is used during performing said therapy prescription process.

22. The method of claim 21, wherein said automatically performing said therapy prescription process comprises:
setting goals and constraints;
assigning goals and constraints; and
assessing goals and constraints for said therapy prescription process.

23. The method of claim 21, further comprising generating a reference image set from said value added at least one image set, wherein said reference image represents a static image of said area of interest prior to any treatment of said area of interest.

24. The method of claim 22, wherein said goals and constraints are selected from the group consisting of: total dose, dose per fraction, a fractionation schedule, identification of whether treatment is complete, definition of anatomical stuctures associated with a disease as well as organs that are not to be unduly irradiated and definition of an anatomical point to be irradiated to a required minimum dose.

25. The method of claim 17, wherein said automatically monitoring comprises laboratory testing of said patient.

26. The method of claim 17, wherein said automatically monitoring comprises physiological measurement of said patient.

27. The method of claim 17, wherein said automatically monitoring comprises clinical observation of said patient.

28. The method of claim 23, further comprising:
automatically generating a reference plan based on said reference image set, wherein said reference plan indicates what is dosimetrically expected for a first fraction of radiation and a total course of radiation delivery to said area of interest; and
automatically generating a positional image set of said patient that includes information of an actual treatment isocenter.

29. The method of claim 28, further comprising assigning goals and constraints to generate said modified therapy based on said positional image set.

30. The method of claim 28, further comprising:
comparing said positional image set with said reference image set; and
automatically modifying a position of said patient based on said comparing.

31. The method of claim 28, further comprising:
automatically determining a positional plan from said positional image set, wherein said positional plan defines dose volume statistics;
automatically comparing said positional plan with said reference plan so as to automatically generate a modified reference plan.
